SEQUENCE LISTING (14 sequences)

SEQ ID NO: 1 - protein, 12 aa, Artificial Sequence
  Synthetic construct
TWYKIAFQRNRK

SEQ ID NO: 2 - protein, 11 aa, Artificial Sequence
  Synthetic construct
WYKIAFQRNRK

SEQ ID NO: 3 - protein, 10 aa, Artificial Sequence
  Synthetic construct
YKIAFQRNRK

SEQ ID NO: 4 - protein, 11 aa, Artificial Sequence
  Synthetic construct
TWYKIAFQRNR

SEQ ID NO: 5 - protein, 10 aa, Artificial Sequence
  Synthetic construct
TWYKIAFQRN

SEQ ID NO: 6 - protein, 9 aa, Artificial Sequence
  Synthetic construct
TWYKIAFQR

SEQ ID NO: 7 - protein, 5 aa, Artificial Sequence
  Synthetic construct
RGDXX

SEQ ID NO: 8 - protein, 5 aa, Artificial Sequence
  Synthetic construct
RGDXX

SEQ ID NO: 9 - protein, 5 aa, Artificial Sequence
  Synthetic construct
RGDXX

SEQ ID NO: 10 - protein, 5 aa, Artificial Sequence
  Synthetic construct
RGDXX

SEQ ID NO: 11 - protein, 5 aa, Artificial Sequence
  Synthetic construct
RGDXX

SEQ ID NO: 12 - protein, 5 aa, Artificial Sequence
  Synthetic construct
RGDXX

SEQ ID NO: 13 - protein, 5 aa, Artificial Sequence
  Synthetic construct
RGDXX

SEQ ID NO: 14 - protein, 5 aa, Artificial Sequence
  Synthetic construct
RGDXX

(19) United States
(12) Patent Application Publication
(10) Pub. No.: US 2005/0261169 A1
(43) Pub. Date: Nov. 24, 2005

(54) Peptide and peptide mimetic derivatives having integrin inhibitor properties III

(76) Inventors: Anja Enderle, Muehltal (DE); Berthold Nies, Frankisch-Crumbach (DE); Horst Kessler, Garching (DE); Jorg Auerheimer, Munchen (DE); Ulrich Hersel, Heidelberg (DE); Claudia Dahmen, Freising (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/858,480

(22) Filed: Jun. 2, 2004

(30) Foreign Application Priority Data
Jun. 2, 2003 (DE) 103 25 049.2

(51) Int. Cl. A61K 38/16; A61K 38/12

(52) U.S. Cl. 514/7; 514/9; 530/317

(57) ABSTRACT

Compounds of the formula I B-Q-X₁ and processes for their preparation, wherein B, Q and X, are as defined in the application, 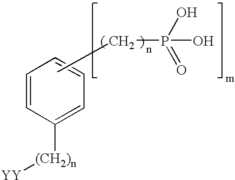 and salts thereof as integrin inhibitors for the treatment of diseases, defects and inflammation caused by implants and of osteolytic diseases, such as osteoporosis, thrombosis, cardiac infarction and arteriosclerosis, and for the acceleration and strengthening of the integration process of the implant or of the biocompatible surface into the tissue.

The invention relates to compounds of the formula I
B-Q-X₁ I
in which
B is a bioactive, cell adhesion-promoting molecule,
Q is absent or is an organic spacer molecule, and
X₁ is an anchor molecule selected from the group consisting of
   —W (i)
   —V—W (ii)
   —V—[V—W₂]₂ (iii) and
   —V—[V—(V—W₂)₂]₂ (iv),
   where W is 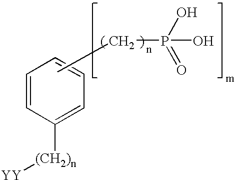
   and V is Lys, Asp or Glu,
m is 1, 2 or 3, and n is in each case, independently of one another, 0, 1, 2, 3, 4, 5, 6, 7 or 8;
YY is an amino or carboxyl group. A free amino group in group B is linked in a peptide-like manner to a free carboxyl group of the spacer molecule Q or of the anchor molecule X₁ or a free amino group of the radical Q is linked in a peptide-like manner to a free carboxyl group of the radical X₁,
and salts thereof.

Similar compounds are disclosed in DE 10040105, DE 19932796, DE 19755800 and DE 19831710.

An object of the invention is novel compounds having valuable properties, in particular those which can be used for the preparation of medicaments.

It has been found that the compounds of the formula I and salts thereof have very valuable pharmacological properties while being well tolerated. In particular, they act as integrin inhibitors, inhibiting, in particular, the interactions of the αᵥβ₃ or αᵥβ₅ integrin receptors with ligands, such as, for example, the bonding of fibrinogen to the β₃ integrin receptor. The compounds exhibit particular efficacy in the case of the integrins αᵥβ₃, αᵥβ₅, α_{IIb}β₃ as well as αᵥβ₁, αᵥβ₆ and αᵥβ₈.

This effect can be demonstrated, for example, by the method described by J. W. Smith et al. in J. Biol. Chem. 265, 12267-12271 (1990).

The dependence of the occurrence of angiogenesis on the interaction between vascular integrins and extracellular matrix proteins is described by P. C. Brooks, R. A. Clark and D. A. Cheresh in Science 264, 569-71 (1994).

The possibility of inhibiting this interaction and thus initiating apoptosis (programmed cell death) of angiogenic vascular cells by a cyclic peptide is described by P. C. Brooks, A. M. Montgomery, M. Rosenfeld, R. A. Reisfeld, T.-Hu, G. Klier and D. A. Cheresh in Cell 79, 1157-64(1994).

Compounds of the formula I which block the interaction of integrin receptors and ligands, such as, for example, of fibrinogen with the fibrinogen receptor (glycoprotein prevent, as GPIIb/IIIa antagonists, the spread of tumor cells by metastasis. This is confirmed by the following observations:

The spread of tumor cells from a local tumor into the vascular system takes place through the formation of microaggregates (microthrombi) through interaction of the tumor cells with blood platelets. The tumor cells are masked by the protection in the microaggregate and are not recognized by the cells of the immune system.

The microaggregates are able to attach themselves to vessel walls, facilitating further penetration of tumor cells into the tissue. Since the formation of the microthrombi is mediated by fibrinogen bonding to the fibrinogen receptors on activated blood platelets, the GPIIa/IIIb antagonists can be regarded as effective metastasis inhibitors.

The phosphonate radical serves to bind the peptides ionically or adsorptively to biocompatible surfaces of, for example, implants which have oxides, such as, for example, surfaces (for example titanium or titanium alloys, such as TiAl₆V₄) or cation-containing surfaces, such as, for example, on amorphous or sintered calcium phosphates (for example hydroxyapatite, bone or teeth) or calcium phosphate cements (for example biocement D).

The invention therefore relates in particular to the compounds of the formula I for ionic or adsorptive bonding to biocompatible surfaces via the functional group of the radical X₁.

The peptides according to the invention facilitate the biofunctionalization of biomaterials, in particular implants for human and animal organs, by coating thereof, stimulating predominantly the adhesion of cell species which are in each case intended to carry out the tissue integration of the corresponding biomaterial. The aim of the use of such coatings is to achieve accelerated and increased integration of various biomaterials/implants having improved long-term stability after introduction thereof into the body.

The peptides according to the invention bind selectively to integrins. After immobilization on biocompatible surfaces, for example implants, they stimulate the adhesion of cells carrying integrins. After coating of the compounds onto the surfaces, the cell species which are also intended to carry out implant integration after implantation in natural tissue can be stimulated selectively to bind. Thus, for example, osteoblasts, osteoclasts and endothelial cells are αᵥ-carrying cell species.

The invention therefore relates to the compounds of the formula I as integrin inhibitors for selective enrichment of cells on implants.

After anchoring to a biocompatible surface, the compounds of the formula I can be employed as medicament active ingredients in human and veterinary medicine, in particular as integrin inhibitors for the treatment of diseases, defects and inflammation caused by implants, such as inadequate and retarded integration of biomaterials and implants, of thrombosis caused by implants, of bone and tooth defects, and of osteolytic diseases, such as osteoporosis, thrombosis, cardiac infarction, arteriosclerosis, in wound healing for supporting the healing process, and for the acceleration and strengthening of the integration process of the implant or of the biocompatible surface into the tissue.

The compounds of the formula I can be employed as antimicrobially active substances in operations where biomaterials, implants, catheters or cardiac pacemakers are used. They have an antiseptic action here. The efficacy of the antimicrobial activity can be demonstrated by the method described by P. Valentin-Weigund et al., in Infection and Immunity, 2851-2855 (1988).

The invention thus relates to the compounds of the formula I as integrin inhibitors for treatment of diseases, defects and inflammation caused by implants and of osteolytic diseases, Such as osteoporosis, thrombosis, cardiac infarction and arteriosclerosis, and for the acceleration and strengthening of the integration process of the implant or of the biocompatible surface into the tissue.

The invention furthermore relates to the use of compounds of the formula I for the preparation of a medicament for the treatment of diseases, defects and inflammation caused by implants and of osteolytic diseases, such as osteoporosis, thrombosis, cardiac infarction and arteriosclerosis, and for the acceleration and strengthening of the integration process of the implant or of the biocompatible surface into the tissue.

Corresponding phosphonate anchor-carrying peptides can be bound ionically to supports having oxide-containing surfaces, such as, for example, implants, affinity chromatographs or microtitre plates, or alternatively to cation-containing surfaces, such as, for example, on amorphous or sintered calcium phosphates (for example hydroxyapatite, bone or teeth) or calcium phosphate cements (for example biocement D).

The invention also relates to the use of compounds of the formula I for coating of implants for human and animal organs by means of ionic or adsorptive bonding.

The abbreviations of amino acid radicals given above and below stand for the radicals of the following amino acids:
Abu 4-aminiobutyric acid
Aha 6-aminiohexanoic acid, 6-aminocaproic acid
Ala alanine
Asn asparagine
Asp aspartic acid
Arg arginine
Cys cysteine
Dab 2,4-diaminobutyric acid
Dap 2,3-diaminopropionic acid
Gln glutamine
Glp pyroglutamic acid
Glu glutamic acid
Gly glycine
His histidine
homo-Phe homo-phenylalanine
Ile isoleucine
Leu leucine
Lys lysine
Met methionine
Nle norleucine
Orn ornithine
Phe phenylalanine
Phg phenylglycine
4-Hal-Phe 4-halophenylalanine
Pro proline
Ser serine
Thr threonine
Trp tryptophan
Tyr tyrosine
Val valine.

Furthermore, the following abbreviations are used below:
Ac acetyl
BOC tert-butoxycarbonyl
CBZ or Z benzyloxycarbonyl
DCCl dicyclohexylcarbodiimide
DMF dimethylformamide
EDCl N-ethyl-N,N′-(dimethylaminopropyl)carbodiimide
Et ethyl
FCA fluoresceincarboxylic acid
FITC fluorescein iosthiocyanate
Fmoc 9-fluorenylmethoxycarbonyl
FTH fluoresceinthiourea
HOBt 1-hydroxybenzotriazole
Me methyl
MBHA 4-methylbenzhydrylamine
Mtr 4-methoxy-2,3,6-trimethylphenylsulfonyl
HATU O-(7-azabenzotriazol-1-yl)-N,N,N′,N′-tetramethyluronium hexafluorophosphate
HONSu N-hydoxysuccinimide
OtBu tert-butyl ester
Oct octanoyl
OMe methyl ester
OEt ethyl ester
POA phenoxyacetyl
Pbf pentamethylbenzofuranyl
Pmc 2,2,5,7,8-pentamethylchroman-6-sulfonyl
Sal salicyloyl
Su Succinyl
TIPS triisopropylsilane
TFA trifluoroacetic acid
TMSBr trimethylsilyl bromide
Trt trityl(triphenylmethyl).

If the above-mentioned amino acids can occur in a plurality of enantiomeric forms, all these forms and also mixtures thereof (for example the DL forms) are included above and below, for example as a constituent of the compounds of the formula I. Furthermore, the amino acids may, for example as a constituent of compounds of the formula I, be provided with corresponding protecting groups known per se. In particular, side-chain modifications of the arginine, as carried out, for example, in the case of non-peptide αᵥβ₃ antagonists (for example by R. Keenan et al., Abstr. Pap. 211 th ACS National Meeting (New Orleans, USA) 1996, MEDI 236), can also be employed in the cyclopeptides, such as, for example, benzimidazole derivatives instead of the guanidine group.

The compounds according to the invention also include so-called prodrug derivatives, i.e. compounds of the formula I modified with, for example, alkyl or acyl groups, sugars or oligopeptides, which are rapidly cleaved in the organism to give the effective compounds according to the invention.

The invention furthermore relates to an implant, suitable for human and animal organs, consisting of a support matrix and a layer of a bioactive, cell adhesion-promoting molecule surrounding this matrix, where the surrounding layer is formed from a compound of the formula I and where an ionic or adsorptive bond exists between the support matrix and this compound. The support matrix and/or the surface thereof preferably contains a metal or metal oxide. The support matrix and/or the surface thereof particularly preferably contains a bone or tooth replacement material, such as, for example, calcium phosphate mixtures.

The invention furthermore relates to a process for the preparation of compounds of the formula I and salts thereof, characterized in that a bioactive molecule B, which may be provided with protecting groups, and a spacer-anchor molecule (Q-X₁) or anchor molecule (X₁) provided with protecting groups are linked to one another in a peptide-like manner, and the protecting groups are subsequently removed, and/or in that a basic or acidic compound of the formula I is converted into one of its salts by treatment with an acid or base.

Above and below, the radicals B, Q and X₁ are as defined for the formula I, unless expressly stated otherwise.
B is preferably a cyclo(Arg-Gly-Asp-Z₁) 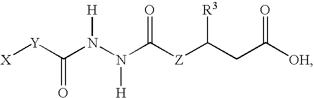
Thr-Trp-Tyr-lys-Ile-Ala-Phe-Gln-Arg-Asn-Arg-Lys,
Trp-Tyr-Lys-Ile-Ala-Phe-Gln-Arg-Asn-Arg-Lys,
Tyr-Lys-Ile-Ala-Phe-Gln-Arg-Asn-Arg-Lys,
Thr-Trp-Tyr-Lys-Ile-Ala-Phe-Gln-Arg-Asn-Arg,
Thr-Trp-Tyr-Lys-Ile-Ala-Phe-Gln-Arg-Asn or a
Thr-Trp-Tyr-Lys-Ile-Ala-Phe-Gln-Arg radical,
   where Z₁, is in each case, independently of one another, an amino acid radical or a di- or tripeptide radical, where the amino acids are selected, independently of one another, from the group consisting of Ala, Asn, Asp, Arg, Cys, Gln, Glu, Gly, His, homo-Phe, Ile, Leu, Lys, Orn, Met, Phe, Phg, Pro, Ser, Thr, Trp, Tyr and Val.

Q is absent or is an organic spacer molecule. This is preferably a [CO—(CH₂)ₓ—NH—]ₘ[CO—CH₂(—O—CH₂CH₂)_{y}—NH—]ₘ, [CO—(CH₂)_{z}—CO—], [NH—(CH₂)_{z}—NH—],[CO—CH₂—(OCH₂CH₂)_{y}—O—CH₂—CO—] or an [NH—CH₂CH₂—(OCH₂CH₂)_{y}—NH—] radical and combinations where m is 1-20, x is 1-12, y is 1-50 and z is 1-12. The above-mentioned compounds which can adopt values of between 1 and 8 for m, values of between 1 and 5 for x and values of between 1 and 6 for y and z have proven particularly advantageous.

X₁ is an anchor molecule, preferably from the group consisting of —W, —V—W, —V[V—W₂]₂ and —V[V—(V—W₂)₂]₂.

The amino acids and amino acid radicals mentioned in the meanings of Z₁ may also derivatized, preference being given to the N-methyl, N-ethyl, N-propyl, N-benzyl and C_{α}-methyl derivatives. Preference is furthermore given to derivatives of Asp and Glu, in particular the methyl, ethyl, propyl, butyl, tert-butyl, neopentyl and benzyl esters of the side-chain carboxyl groups, furthermore also derivatives of Arg, which may be substituted by an acetyl, benzoyl, methoxycarbonyl or ethoxycarbonyl radical on the —NH—C(═NH)—NH₂ group.

X is preferably H₂N—C(═NH)—NH—, Het-NH—, H₂N—C(═NH)—, A-C(═NH)—NH— or a Het radical.

Y is preferably a —(CH₂)ₙ—, 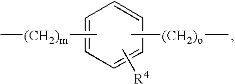
—(CH₂)ₛ—CH(R⁴)—(CH₂)ₜ— or —(CH₂)ₚ-Het¹—(CH₂)_{q}-radical.

Z is preferably N—R² or CH—R², where R² can preferably be an H atom or an alkyl radical having from 1 to 4 carbon atoms.

R³ is preferably an H atom or an Ar, Het or A radical, where A, Ar and Het have on of the meanings indicated above or below.

R⁴ is preferably an H atom or an A, Ar, OH, OA, OAr, arylalkyl, Hal, CN, NO₂, CF₃ OCF₃ radical. Arylalkyl is preferably benzyl, phenylethyl, phenylpropyl or naphthylmethyl, particularly preferably benzyl.

A is preferably a COOH, NH₂ or alkyl radical having from 1 to 6 carbon atoms, unsubstituted or substituted by COOH or NH₂. A is preferably methyl, furthermore ethyl, propyl, n-butyl, isobutyl, sec-butyl or tert-butyl, furthermore also n-pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, or 1,1,2- or 1,2,2-trimethylpropyl. A is particularly preferably methyl.

Ar is preferably phenyl which is unsubstituted or mono-, di- or trisubstituted by A, OH, OA CF₃, OCF₃, CN, NO₂ or Hal and which may be substituted by phenyl which is mono-, di- or trisubstituted by A, OH, OA, NH₂, OCF₃, CN, NO₂ or Hal, in such a way as to give unsubstituted or substituted biphenyl.

Ar is therefore preferably phenyl, o-, m- or p-methylphenyl, o-, m- or p-ehtylphenyl, o-, m- or p-propylphenyl, o-, m- or p-isopropylphenyl, o-, m- or p-tert-butylphenyl, o-, m- or p-hydroxyphenyl, o-, m- or p-methoxyphenyl, o-, m- or p-ethoxyphenyl, o-, m- or p-trifluoromethylphenyl, o-, m- or p-trifluoromethoxyphenyl, o-, m- or p-fluorophenyl, o-, m- or p-chlorophenyl, o-, m- or p-bromophenyl, o-, m- or p-nitrophenyl, or o-, m- or p-aminomethylphenyl.

Het is a saturated or partially or fully unsaturated mono- or bicyclic heterocyclic radical having from 5 to 10 ring members, where from 1 to 3 N atoms and/or 1 S or O atom may be present and the heterocyclic radical may be mono- or disubstituted by CN, Hal, OH, NH₂,COOH, OA, CF₃, A, NO₂, Ar or OCF₃.

Het is preferably o-, m- or p-substituted pyridyl, 2-, 4-, 5- or 6-substituted pyrimidyl or 3-, 4-, 5- or 6-substituted pyridazyl, each of which is preferably unsubstituted or substituted by a methyl, ethyl or propyl group or a methylamino, ethylamino or propylamino group (relates to all of the three heteroaromatic radicals mentioned), or 2-substituted benzimidazolyl, which is unsubstituted or substituted by a 3-methyl, 3-ethyl or 3-benzyl group, or 2-substituted dihydroimidazolyl, tetrahydropyrimidyl or tetrahydropyridyl.

Examples which are preferably present in Het are:

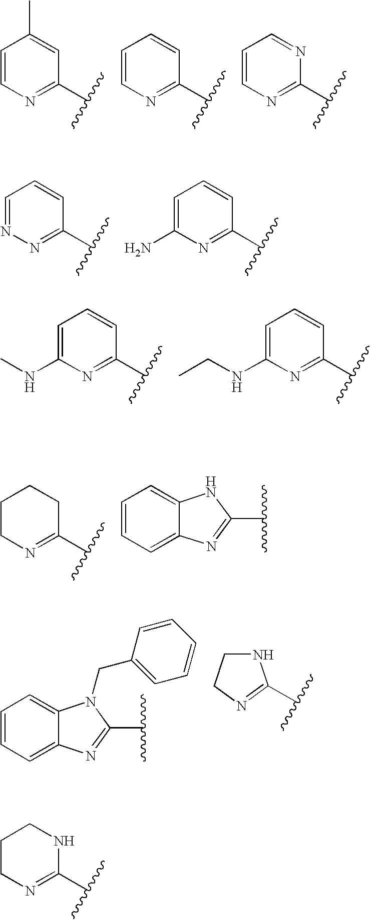

Het¹ is a 5- or 6-membered aromatic heterocyclic ring having from 1 to 4 N, O and/or s atoms, which may be unsubstituted or mono- or disubstituted by F, Cl, Br, A, OA or OCF₃.

Het¹ is preferably 2,4-, 3,5- or 2,5-disubstituted pyridyl or 2,4-, 2,5-, 2,6- or 4,6-disubstituted pyrimidyl, 2,4- or 2,5-disubstituted 1,3-oxazolyl or 1,3-thiazolyl.

OA is preferably methoxy, ethoxy, propoxy or butoxy, furthermore also pentyloxy or hexyloxy.

Hal is preferably F, Cl or Br, but also I.

The indices n is 4, 5 or 6; m, o, p and q are, each independently, 0, 1 or 2; and s and t are 0, 1, 2, 3, 4 or 5, unless expressly stated otherwise.

The compounds of the formula I can have one or more centers of chirality and can therefore occur in various stereoisomeric forms. The formula I covers all these forms.

Accordingly, the invention relates in particular to the compounds of the formula I in which at least one of the said radicals has one of the preferred meanings indicated above.

Particular preference is given to the following compounds of the formula I:
a) cyclo(Arg-Gly-Asp-D-Phe-Lys(^{ε}NH—[CO—(CH₂)₅—NH]₂-Lys-(CO—C₆H₃(CH₂PO₃H₂)₂)₂))
b) cyclo(Arg-Gly-Asp-D-Phe-Lys(^{ε}NH—[CO—(CH₂)₅—NH]₂—(CO—C₆H₃(CH₂PO₃H₂)₂)₂))
c) cyclo(Arg-Gly-Asp-D-Phe-Lys(^{ε}NH—[CO—(CH₂)₅—NH]₃-Lys-(CO—C₆H₃(CH₂PO₃H₂)₂)₂))
d) cyclo(Arg-Gly-Asp-D-Phe-Lys(^{ε}NH—[CO—(CH₂)₅—NH]₃—(CO—C₆H₃(CH₂PO₃H₂)₂)₂))
e) cyclo(Arg-Gly-Asp-D-Phe-Lys(^{ε}NH—[CO—(CH₂)₅—NH]₄-Lys-(CO—C₆H₃(CH₂PO₃H₂)₂)₂))
f) cyclo(Arg-Gly-Asp-D-Phe-Lys(^{ε}NH—[CO—(CH₂)₅—NH]₄—(CO—C₆H₃(CH₂PO₃H₂)₂)₂))
g) cyclo(Arg-Gly-Asp-D-Phe-Lys(^{ε}NH—[CO—CH₂(—O—CH₂CH₂)₆—NH]₂-Lys-(CO—C₆H₃(CH₂PO₃H₂)₂)₂))
h) cyclo(Arg-Gly-Asp-D-Phe-Lys(^{ε}NH—[(O—CH₂CH₂)₆—NH]₂-Lys-(CO—C₆H₃(CH₂PO₃H₂)₂)₂))
i) cyclo(Arg-Gly-Asp-D-Phe-Lys(^{ε}NH—[CO—(CH₂)₅—NH]₂-Lys-[Lys-(CO—C₆H₃(CH₂PO₃H₂)₂)₂]₂))
j) cyclo(Arg-Gly-Asp-D-Phe-Lys(^{ε}NH—[CO—(CH₂)₅—NH]₃-Lys-[Lys-(CO—C₆H₃(CH₂PO₃H₂)₂)₂]₂))
k) cyclo(Arg-Gly-Asp-D-Phe-Lys(^{ε}NH—[CO—CH₂(—O—CH₂CH₂)₆—NH]₂-Lys-[Lys-(CO—C₆H₃(CH₂PO₃H₂)₂)₂]₂)).

The compounds of the formula I and also the starting materials for their preparation are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants which are known per se, but are not mentioned here in greater detail.

If desired, the starting materials can also be formed in situ by not isolating them from the reaction mixture, but instead immediately converting them further into the compounds of the formula I.

The fragment coupling or the coupling of ligand to linker is generally carried out in an inert solvent, where a carboxylic acid fragment (for example phosphonate linker HO—[CO—(CH₂)₅—NH]₄-Lys-[Lys-(CO—C₆H₃(CH₂—PO₃H₂)₂₂] is dissolved in DMF with HATU, HOAt and 2,4,6-collidine, and an amine fragment (cyclopeptide, for example c[R(Pbf)G(OtBu)fK]) is added.

Suitable inert solvents are, for example, hydrocarbons, such as hexane, petroleum ether, benzene, toluene or xylene; chlorinated hydrocarbons, such as trichloroethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, N-methylpyrrolidone, dimethylacetamide or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, water, or mixtures of the said solvents.

Cyclic compounds can be prepared by cyclization of the linear compounds, as described, for example, in DE 43 10 643, in Houben-Weyl, l.c., Volume 15/II, pages 1 to 806 (1974), or by S. Zimmer, E. Hoffmann, G. Jung and H. Kessler, Liebigs Ann. Chem. 1993, 497-501.

The linear peptides can be synthesized, for example, as described by R. B. Merrifield, Angew. Chemie 1985, 97, 801-812.

Open-chain linear compounds, such as, for example, compounds of the formula I, can, in addition, be prepared by conventional methods of amino acid and peptide synthesis, for example also by the Merrifield solid-phase synthesis (see also, for example, B. F. Gysin and R. B. Merrifield, J. Am. Chem. Soc. 94, 3102 ff (1972)).

The compounds of the formula I can furthermore be obtained by liberating them from their functional derivatives by solvolysis, in particular hydrolysis, or by hydrogenolysis.

Preferred starting materials for the solvolysis or hydrogenolysis are those which contain corresponding protected amino and/or hydroxyl groups instead of one or more free amino and/or hydroxyl groups, preferably those which carry an amino-protecting group instead of an H atom bonded to an N atom, for example those which conform to the formula I, but contain an NHR′ group (in which R′ is an amino-protecting group, for example BOC or CBZ) instead of an NH₂ group.

Preference is furthermore given to starting materials which carry a hydroxyl-protecting group instead of the H atom of a hydroxyl group, for example those which conform to the formula I, but contain an R″O-phenyl group (in which R″ is a hydroxyl-protecting group) instead of a hydroxyphenyl group.

It is also possible for a plurality of—identical or different—protected amino and/or hydroxyl groups to be present in the molecule of the starting material. If the protecting groups present are different from one another, they can in many cases be cleaved off selectively.

The term “amino-protecting group” is known in general terms and relates to groups which are suitable for protecting (blocking) an amino group against chemical reactions, but are easy to remove after the desired chemical reaction has been carried out elsewhere in the molecule. Typical of such groups are, in particular, unsubstituted or substituted acyl, aryl, aralkoxylmethyl or aralkyl groups. Since the amino-protecting groups are removed after the desired reaction (or reaction sequence), their type and size are furthermore not crucial; however, preference is given to those having 1-20, in particular 1-8, carbon atoms. The term “acyl group” is to be understood in the broadest sense in connection with the present process. It includes acyl groups derived from aliphatic, araliphatic, aromatic or heterocyclic carboxylic acids or sulfonic acids, and, in particular, alkoxycarbonyl, aryloxycarbonyl and especially aralkoxycarbonyl groups. Examples of such acyl groups are alkanoyl, such as acetyl, propionyl and butyryl; aralkanoyl, such as phenylacetyl; aroyl, such as benzoyl and tolyl; aryloxyalkanoyl, such as POA; alkoxycarbonyl, such as methoxcarbonyl, ethoxycarbonyl, 2,2,2-trichloroethoxycarbonyl, BOC and 2-iodoethoxycarbonyl; aralkoxycarbonyl, such as CBZ (“carbobenzoxy”), 4-methoxybenzyloxycarbonyl and FMOC; and arylsulfonyl, such as Mtr, Pbf and Pmc. Preferred amino-protecting groups are BOC and Mtr, furthermore CBZ, Fmoc, benzyl and acetyl.

The term “hydroxyl-protecting group” is likewise known in general terms and relates to groups which are suitable for protecting a hydroxyl group against chemical reactions, but are easy to remove after the desired chemical reaction has been carried out elsewhere in the molecule. Typical of such groups are the above-mentioned unsubstituted or substituted aryl, aralkyl or acyl groups, furthermore also alkyl groups. The nature and size of the hydroxyl-protecting groups are not crucial since they are removed again after the desired chemical reaction or reaction sequence; preference is given to groups having 1-20, in particular 1-10, carbon atoms. Examples of hydroxyl-protecting groups are, inter alia, benzyl, p-nitrobenzoyl, p-toluenesulfonyl, tert-butyl and acetyl, where benzyl and tert-butyl are particularly preferred. The COOH groups in aspartic acid and glutamic acid are preferably protected in the form of their tert-butyl esters (for example Asp(OtBu)).

The compounds of the formula I are liberated from their functional derivatives—depending on the protecting group used—for example using strong acids, advantageously using TFA or perchloric acid, but also using other strong inorganic acids, such as hydrochloric acid or sulfuric acid, strong organic carboxylic acids, such as trichloroacetic acid, or sulfonic acids, such as benzene- or p-toluenesulfonic acid. The presence of an additional inert solvent is possible, but is not always necessary. Suitable inert solvents are preferably organic, for example carboxylic acids, such as acetic acid, ethers, such as tetrahydrofuran or dioxane, amides, such as DMF, halogenated hydrocarbons, such as dichloromethane, furthermore also alcohols, such as methanol, ethanol or isopropanol, and water mixtures of the above-mentioned solvents are furthermore suitable. TFA is preferably used in excess without addition of a further solvent, and perchloric acid is preferably used in the form of a mixture of acetic acid and 70% perchloric acid in the ratio 9:1. The reaction temperatures for the cleavage are advantageously between about 0 and about 50°, preferably between 15° and 30° (room temperature).

The BOC, OBut, Pbf, Pmc and Mtr groups can, for example, preferably be cleaved off using TFA in dichloromethane or using approximately 3 to 5N HCl in dioxane at 15-30°, and the FMOC group can be cleaved off using an approximately 5 to 50% solution of dimethylamine, diethylamine or piperidine in DMF at 15-30°.

The trityl group is employed to protect the amino acids histidine, asparagine, glutamine and cysteine. They are cleaved off, depending on the desired end product, using TFA/10% thiophenol, with the trityl group being cleaved off from all the said amino acids; on use of TFA/anisole, TFA/thioanisole or TFA/TIPS/H₂O, only the trityl group of His, Asn and Gln is cleaved off, whereas it remains on the Cys side chain.

The Pbf (pentamethylbenzofuranyl) group is employed to protect Arg. It is cleaved off using, for example, TFA in dichloromethane.

Hydrogenolytically removable protecting groups (for example CBZ or benzyl) can be cleaved off, for example, by treatment with hydrogen in the presence of a catalyst (for example a noble-metal catalyst, such as palladium, advantageously on a support, such as carbon). Suitable solvents here are those indicated above, in particular, for example, alcohols, Such as methanol or ethanol, or amides, such as DMF. The hydrogenolysis is generally carried out at temperatures between about 0 and 100° and pressures between about 1 and 200 bar, preferably at 10-30° and 1-10 bar. Hydrogenolysis of the CBZ group succeeds well, for example, on 5 to 10% Pd/C in methanol or using ammonium fonnate (instead of hydrogen) on Pd/C in methanol/DMF at 10-30°.

A base of the formula I can be converted into the associated acid-addition salt using an acid, for example by reaction of equivalent amounts of the base and the acid in an inert solvent, such as ethanol, followed by evaporation. Suitable acids for this reaction are, in particular, those which give physiologically acceptable salts. Thus, it is possible to use inorganic acids, for example sulfuric acid, nitric acid, hydrohalic acids, such as hydrochloric acid or hydrobromic acid, phosphoric acids, such as orthophosphoric acid, or sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, pivalic acid, diethylacetic acid, malonic acid, succinic acid, pimelic acid, fumaric acid, maleic acid, lactic acid, tartaric acid, malonic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, naphthalenemono- and -disulfonic acids, and laurylsulfuric acid. Salts with physiologically unacceptable acids, for example picrates, can be used for the isolation and/or purification of the compounds of the formula I.

On the other hand, an acid of the formula I can be converted into one of its physiologically acceptable metal or ammonium salts by reaction with a base. Suitable salts here are, in particular, the sodium, potassium, magnesium, calcium and ammonium salts, furthermore substituted ammonium salts, for example the dimethyl-, diethyl- or diisopropyl-ammonium salts, monoethanol-, diethanol- or diisopropanolammonium salts, cyclohexyl-, dicyclohexylammonium salts, dibenzylethylenediammonium salts, furthermore, for example, salts with arginine or lysine.

Above and below, all temperatures are given in ° C. In the following examples, “conventional work-up” means that water is added if necessary, the pH is adjusted, if necessary, to between 2 and 10, depending on the constitution of the end product, the mixture is extracted with ethyl acetate or dichloromethane, the phases are separated, the organic phase is dried over sodium sulfate and evaporated, and the product is purified by chromatography on silica gel and/or by crystallization. Rf values on silica gel; eluent: ethyl acetate/methanol 9:1.

RT=retention time (minutes) on HPLC in the following systems:

| | |
|---|---|
| [A] | |
| Column: | YMC ODS A RP 5C₁₈, 250 × 4.6 mm |
| Eluent A: | 0.1% TFA in water |
| Eluent B: | 0.1% TFA in acetonitrile |
| Flow rate: | 1 ml/min |
| Gradient: | 0-50% B/30 min. |
| [B] | |
| as for [A]; | |
| Gradient: | 5-50% B/30 min. |
| [C] | |
| as for [A]; | |
| Gradient: | 10-50% B/30 min. |
| Mass spectrometry (MS): | EI (electron impact ionisation) M⁺ |
| | FAB (fast atom bombardment) (M + H)⁺ |
| | ESI (electrospray ionisation) (M + H)⁺ |

DMPP resin stands for 4-(2′,4′-dimethoxyphenylhydroxymethyl)phenoxy resin, which allows, for example, the synthesis of side-chain-protected peptides, TCP resin denotes trityl chloride-polystyrene resin.

The following examples describe firstly fragment coupling and the cleavage of phosphonic esters and secondly the synthesis of selected cyclopeptide derivatives containing phosphonate linkers. The process for coating the various metal or bone replacement material moldings is explained in greater detail with reference to Examples 7 to 9.

### EXAMPLE 1

### Fragment Coupling in Solution

0.2 mmnol of carboxylic acid fragment (for example phosphonate linker HO—[CO—(CH₂)₅—NH]₄-Lys-(CO—C₆H₃(CH₂PO₃H₂)₂)₂), 0.98 eq of HATU, 1.1 eq of HOAt and 10 eq of 2,4,6-collidine are dissolved in 2 ml of DMF. After 1.5 hours, 1 eq of amine fragment (for example cyclopeptide c[R(Pbf)G(OtBu)fK]) is added. The mixture is stirred at room temperature for 24 hours, and the product is purified by preparative HPLC.

### EXAMPLE 2

### Cleavage of Phosphonic Esters in the Phosphonate Linkers

The cleavage of the phosphonate ester groups is carried out at the same time as the removal of the side-chain-protecting groups of the peptide or peptide mimetic using 90% TFA, 5% H₂O and 5% TIPS. After 4 hours, the solvent is stripped off, and the residue is taken up in acetic acid and precipitated in cold diethyl ether. The precipitate is separated off and lyophilized from H₂O.

### EXAMPLE 3

### Synthesis of the Phosphonate Linkers

The phosphonate linkers were synthesized in a solid-phase peptide synthesis by the Fmoc strategy (see G. B. Fields, R. L. Nobie, Int. J. Pept. Protein Res. 1990, 35, 161-214).

The final unit to be coupled was tetrabenzyl 5-carboxy-m-xylenebisphosphonate.

Synthesis of tetrabenzyl 5-carboxy-m-xylenebisphiosphonate:

### Methyl 3,5-bis(bromomethyl)benzoate

30 mmol of methyl 3,5-(bismethyl)benzoate (5.0 g) are dissolved in 50 ml of CCl₄. After addition of 2 eq of N-bromosuccinimide (10.6 g) and 150 mg of benzoyl peroxide, the mixture is refluxed for 3 hours. The cooled mixture is filtered, and the solvent is stripped off. The product is obtained as an oil (10.5 g), which can be brought to crystallization by covering with 100 ml of hexane. 3.3 g of pale-yellow solid are obtained.

### Tetrabenzyl 5-methoxycarbonyl-m-xylenebisphosphonate

4.7 mmol of methyl 3,5-bis(bromomethyl)benzoate (1.5 g) are suspended in 3 eq of tribenzyl phosphite (4.9 g). The mixture is heated in an oil bath at 140° C. for 3 hours, while benzyl bromide formed is stripped off in a high vacuum. After cooling, the residue is separated by chromatography on 250 g of silica gel with ethyl acetate as eluent. The product is obtained as an oil (1.76 g).

### Tetrabenzyl 5-carboxy-m-xylenebisphosphonate

1.5 mmol of tetrabenzyl 5-methoxycarbonyl-m-xylenebisphosphonate (1.0 g) are dissolved in methanol and water 2:1 and stirred with 1.5 eq of lithium hydroxide (53 mg) at room temperature for four days. The pH of the solution is subsequently adjusted to 2.5 using 1 N hydrochloric acid, and the methanol is stripped off. The product is extracted with ethyl acetate. Stripping-off of the solvent gives 0.97 g of oil.

Ti or TiAl₆V₄ moldings having a diameter of 10 mm and a height of 1-2 mm are cleaned.

The moldings are transferred into 48-well plates (Costar, “non-tissue culture treated” Art. No. 3574). For bonding of the bioactive, cell adhesion-promoting molecules B (where B can be a cyclopeptide, peptide mimetic or linear peptide) to the prepared moldings, stock solutions containing B molecules (“B solutions”) are prepared in a final concentration of 1 mM in an aqueous buffer. Concentration series with the “B solution” final concentrations of 1 nM, 10 nM, 100 nM, 1 μM, 10 μM and 100 μM in each case are subsequently prepared by dilution with buffer. The moldings are covered with 250 μl of the respective B solutions in each case and subsequently incubated at room temperature for 18-24 hours. For removal of unbound B molecules, the samples are washed three times with buffer and stored in buffer overnight.

Nonspecific cell binding sites are blocked by addition of in each case 250 μl/molding of a 5% BSA (bovine serum albumin) solution, pH 7.4, followed by incubation at room temperature for 2 hours and washing once with buffer.

Ti or TiAl₆V₄ moldings treated not with B solutions, but with corresponding buffer solutions (TRIS HCl 10 mM, pH 8.7; TRIS HClO₄ 10 mM, pH 8.7; PBS, pH 7.4) function as negative controls.

The degree of resultant coating on the moldings is assessed analytically and the biological efficacy determined by a cell adhesion test in vitro.

### EXAMPLE 4

Calcium phosphate-based moldings are cleaned.

For coating of the moldings with “B solution”, the procedure described under Example 3 is followed.

The degree of resultant coating on the moldings is assessed analytically and the biological efficacy determined by a cell adhesion test in vitro.

Example of ELISA Test:

The amount of bound peptide on the surface can be determined by means of an RGD-specific antibody.

Example of Cell Adhesion Test:

The adhesion of mouse MC3T3 E1 osteoblast cultures to RGD-peptide-coated titanium surfaces in vitro was investigated. 50,000 cells/cm² were seeded, and the proportion of adhered cells was determined after incubation for one hour in serum-free medium at 37° C./95% atmospheric humidity.
Cell adhesion rate [%]=adhered cells/seeded cells×100
Peptide: cell adhesion rate [%]
The results can be found in the figures.

### BRIEF DESCRIPTION OF THE DRAWINGS

FIG. 1: Results of the ELISA test

FIG. 2: Results of the cell adhesion test

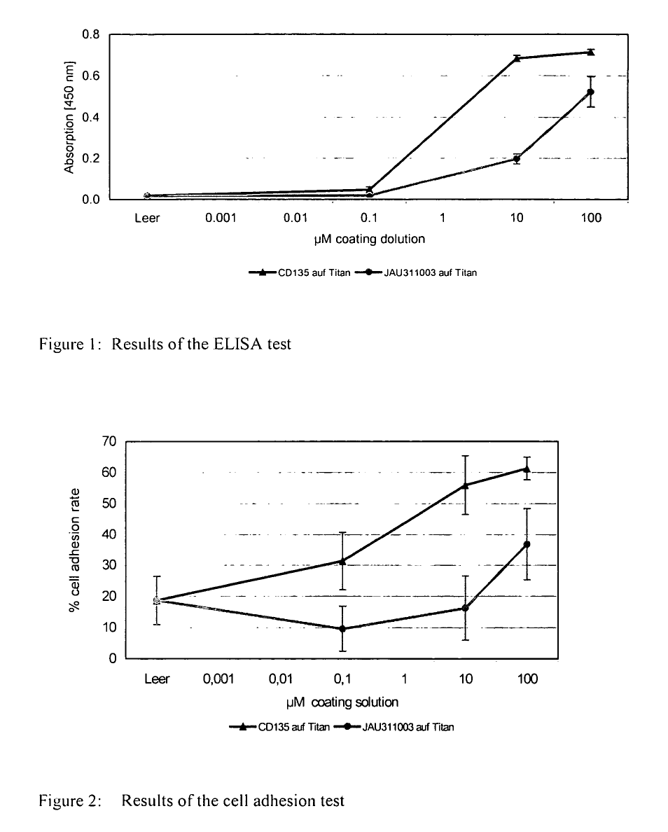

The abbreviations shown in the figures have the following meanings:
CD135 is cyclo(Arg-Gly-Asp-D-Phe-Lys(^{ε}NH—[CO—(CH₂)₅—NH]₃—(CO—C₆H₃(CH₂PO₃H₂)₂)) and JAU311003 is cyclo(Arg-Gly-Asp-D-Phe-Lys(^{ε}NH—[CO—(CH₂)₅—NH]₃—(CO—C₆H₃(CH₂PO₃H₂)₂)₂)).

It can be seen in the ELISA that more peptide is bound to the surface in the case of compound CD 135, which results in the cell adhesion test in a higher cell adhesion rate for all concentrations measured.

The cell adhesion at the beginning (blank) corresponds to the cell adhesion for uncoated titanium plates. Coating of the plates with a 100 μM coating solution enables the cell adhesion to be tripled compared with uncoated plates.

The entire disclosure of all applications, patents and publications, cited herein and of corresponding German application No. 10325049.2, filed Jun. 2, 2003, is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A compound of formula I
B-Q-X₁ I
in which
B is a bioactive cell adhesion-promoting group,
Q is absent or is an organic spacer molecule, and
X₁ is
—W (i)
—V—W (ii)
—V—[V—W₂]₂ (iii) or
—V—[V—(V—W₂)₂]₂ (iv),
W is 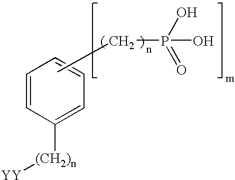
V is Lys, Asp or Glu,
m is 1, 2 or 3,
n is, in each case independently of one another, 0, 1, 2, 3, 4, 5, 6, 7 or 8, and
YY is an amino or carboxyl group,
wherein a free amino group in group B is linked in a peptide-like manner to a free carboxyl group in group Q or in group X₁, or a free amino group in group Q is linked in a peptide-like manner to a free carboxyl group in group X₁, or a salt thereof.

2. A compound according to claim 1, in which group B
cyclo(Arg-Gly-Asp-Z₁) (iv), 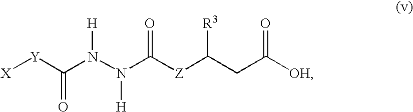
Thr-Trp-Tyr-Lys-Ile-Ala-Phe-Gln-Arg-Asn-Arg-Lys (vi),
Trp-Tyr-Lys-Ile-Ala-Phe-Gln-Arg-Asn-Arg-Lys (vii),
Tyr-Lys-Ile-Ala-Phe-Gln-Arg-Asn-Arg-Lys (viii),
Thr-Trp-Tyr-Lys-Ile-Ala-Phe-Gln-Arg-Asn-Arg (ix),
Thr-Trp-Tyr-Lys-Ile-Ala-Phe-Gln-Arg-Asn (x), or
Thr-Trp-Tyr-Lys-Ile-Ala-Phe-Gln-Arg (xi),
wherein
Z₁ is, in each case independently of one another, an amino acid radical or a di- or tripeptide radical, where the amino acid(s) are, independently of one another, Ala, Asn, Asp, Arg, Cys, Gln, Glu, Gly, His, homo-Phe, Ile, Leu, Lys, Met, Orn, Phe, Phg, Pro, Ser, Thr, Trp, Tyr or Val,
x is H₂N—C(═NH)—NH—, Het-NH—, H₂N—C(═NH)—, A-C(═NH)—NH— or Het-,
Y is —(CH₂)ₙ—, 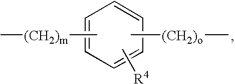
—(CH₂)ₛ—(R⁴)—(CH₂)ₜ— or —(CH₂)ₚ-Het¹-(CH₂)_{q}—,
Z is N—R² or CH—R²,
R² is H or alkyl having 1 to 4 carbon atoms,
R³ is H, Ar, Het or A,
R⁴ is H, A, Ar, OH, OA, OAr, arylalkyl, Hal, CN, NO₂, CF₃ or OCF₃,
A is COOH, NH₂ or alkyl having 1-6 carbon atoms, which is unsubstituted or substituted by COOH and/or NH₂,
Ar is phenyl, which is unsubstituted or mono-, di- or trisubstituted by A, OH, OA, CF₃, OCF₃, CN, NO₂ or Hal, and which is optionally substituted by phenyl, which is mono-, di- or trisubstituted by A, OH, OA, NH₂, OCF₃, CN, NO₂ or Hal, in such a way as to give unsubstituted or substituted biphenyl,
Hal is F, Cl, Br or I,
Het is a saturated or partially or fully unsaturated mono- or bicyclic heterocyclic radical having 5 to 10 ring members, wherein 1 to 3 N atoms and/or 1 S or O atom are present, and which is optionally mono- or disubstituted by CN, Hal, OH , NH₂, COOH, OA, CF₃, A, NO₂, Ar or OCF₃,
Het¹ is a 5- or 6-membered aromatic heterocyclic ring having 1 to 4 N and/or S atoms, which is optionally unsubstituted or mono- or disubstituted by F, Cl, Br, A, OA or OCF₃,
n is 4, 5 or 6,
m, o, p and q are, each independently, 0, 1 or 2, and
s and t are, each independently, 0, 1, 2, 3, 4 or 5.

3. A compound according to claim 1, in which Q is
[CO—(CH₂)ₓ—NH—]ₘ (xii),
[CO—CH₂ (—O—CH₂CH₂)_{y}—NH—]ₘ (xiii),
[CO—(CH₂)_{z}—CO—] (xiv),
[NH—(CH₂)_{z}—NH—] (xv),
[CO—CH₂—(OCH₂CH₂)_{y}—O—CH₂—CO—] (xvi), or
[NH—CH₂CH₂—(OCH₂CH₂)_{y}—NH—] (xvii),
or a combination thereof,
in which
m is, 1-20,
x is 1-12,
y is 1-50, and
z is 1-12.

4. A compound according to claim 1, in which Q is
[CO—(CH₂)ₓ—NH—]ₘ (xii),
[CO—CH2(—O—CH₂CH₂)_{y}—NH—]ₘ (xiii),
[CO—(CH₂)_{z}—CO—] (xiv),
[NH—(CH₂)_{z}—NH—] (xv),
[CO—CH₂—(OCH₂CH₂)_{y}—O—CH₂—CO—] (xvi), or
[NH—CH₂CH₂—(OCH₂CH₂)_{y}—NH—] (xvii),
or a combination thereof,
in which
m is 1-8,
x is 1-5,
y is 1-6, and
z is 1-6.

5. A compound according to claim 1, which is
a) cyclo(Arg-Gly-Asp-D-Phe-Lys(⁶⁸NH—[CO—(CH₂)₅—NH]₂-Lys-(CO—C₆H₃(CH₂PO₃H₂)₂)₂)),
b) cyclo(Arg-Gly-Asp-D-Phe-Lys(^{ε}NH—[CO—(CH₂)₅—NH]₂—(CO—C₆H₃(CH₂PO₃H₂)₂)₂)),
c) cyclo(Arg-Gly-Asp-D-Phe-Lys(^{ε}NH—[CO—(CH₂)₅—NH]₃-Lys-(CO—C₆H₃(CH₂PO₃H₂)₂)₂)),
d) cyclo(Arg-Gly-Asp-D-Phe-Lys(^{ε}NH—[CO—(CH₂)₅—NH]₃—(CO—C₆H₃(CH₂PO₃H₂)₂)₂)),
e) cyclo(Arg-Gly-Asp-D-Phe-Lys(^{ε}NH—[CO—(CH₂)₅—NH]₄-Lys-(CO—C₆H₃(CH₂PO₃H₂)₂)₂)),
f) cyclo(Arg-Gly-Asp-D-Phe-Lys(^{ε}NH—[CO—(CH₂)₅—NH]₄—(CO—C₆H₃(CH₂PO₃H₂)₂)₂)),
g) cyclo(Arg-Gly-Asp-D-Phe-Lys(^{ε}NH—[CO—CH₂(—O—CH₂CH₂)₆—NH]₂-Lys-(CO—C₆H₃(CH₂PO₃H₂)₂)₂)),
h) cyclo(Arg-Gly-Asp-D-Phe-Lys(^{ε}NH—[(—O—CH₂CH₂)₆—NH]₂-Lys-(CO—C₆H₃(CH₂PO₃H₂)₂)₂)),
i) cyclo(Arg-Gly-Asp-D-Phe-Lys(^{ε}NH—[CO—(CH₂)₅—NH]₂-Lys-[Lys-(CO—C₆H₃(CH₂PO₃H₂)₂)₂]₂)),
j) cyclo(Arg-Gly-Asp-D-Phe-Lys(^{ε}NH—[CO—(CH₂)₅—NH]₃-Lys-[Lys-(CO—C₆H₃(CH₂PO₃H₂)₂)₂]₂)), or
k) cyclo(Arg-Gly-Asp-D-Phe-Lys(^{ε}NH—[CO—CH₂(O—CH₂CH₂)₆—NH]₂-Lys-[Lys-(CO—C₆H₃(CH₂PO₃H₂)₂)₂]₂)).

6. A pharmaceutical composition comprising a compound according to claim 1 and a pharmaceutically acceptable carrier.

7. An implant, suitable for use in a human or in an animal, comprising a support matrix and a layer surrounding the support matrix, said layer comprising a compound according to claim 1, wherein an ionic or adsorptive bond exists between the support matrix and the layer.

8. An implant according to claim 7, wherein the support matrix and/or the surface thereof is a metal or a metal oxide.

9. An implant according to claim 7, wherein the support matrix and/or the surface thereof is a bone or tooth replacement material.

10. An implant according to claim 9, wherein the bone or tooth replacement material comprises a calcium phosphate mixture.

11. Process for preparing a compound according to claim 1 or a salt thereof, comprising linking together in a peptide-like manner a molecule B, which optionally is protected by a protecting group, and a molecule Q-X₁ or X₁, which is protected by a protecting group, followed by removing the protecting group(s), and optionally a basic or acidic compound of formula I is converted into one of its salts by treatment with an acid or base.

12. A method for treating a disease, defect or inflammation caused by an implant, an osteolytic disease, or accelerating and/or strengthening the integration process of an implant or of a biocompatible surface into tissue, comprising administering a compound of claim 1 to a patient in need thereof.

13. A method for coating an implant, comprising forming an ionic or adsorptive bond between said implant and a compound according to claim 1.

14. A method for treating osteoporosis, thrombosis, cardiac infarction or arteriosclerosis, comprising administering a compound of claim 1 to a patient in need thereof.
